(19) United States
(12) Patent Application Publication
(10) Pub. No.: US 2024/0375096 A1
(43) Pub. Date: Nov. 14, 2024

(54) DETECTION STRUCTURE AND METHOD, DETECTION CHIP, AND SENSING DEVICE

(71) Applicant: BGI SHENZHEN, SHENZHEN, GUANGDONG (CN)

(72) Inventors: Quanxin YUN, SHENZHEN, GUANGDONG (CN); Zhaohui WANG, SHENZHEN, GUANGDONG (CN); Yuxiang LI, SHENZHEN, GUANGDONG (CN); Ao CHEN, SHENZHEN, GUANGDONG (CN); Wenwei ZHANG, SHENZHEN, GUANGDONG (CN); Xun XU, SHENZHEN, GUANGDONG (CN)

(21) Appl. No.: 18/687,941

(22) PCT Filed: Aug. 31, 2021
(86) PCT No.: PCT/CN2021/115778

§ 371 Date: Feb. 29, 2024

(51) Int. Cl. B01L 3/00; C12Q 1/6869

(52) U.S. Cl. CPC B01L 3/502; C12Q 1/6869; B01L 2200/10; B01L 2300/0645; B01L 2300/18

(57) ABSTRACT

Provided are a detection structure and method, a detection chip, and a sensing device. The detection structure (200) includes a sensing device (100) at least including a detection chip (000), a fluid tank (101), and a carrier plate (102), and a detector (201) configured to capture and analyze a signal generated in the sensing device (100). The fluid tank (101) is disposed at the carrier plate (102) and forms a cavity (103) with the carrier plate (102). The detection chip (000) is located in the cavity (103) and at least includes a substrate (001), a first electrode (002) and a first circuit (003) that are disposed at the substrate. The first electrode (002) is connected to a second electrode (004) through the first circuit (003) to form an electrical circuit.

### FIELD

The present disclosure is related to the field of biotechnologies, and more particularly, to a detection structure and method, a detection chip, and a sensing device.

### BACKGROUND

As a commonly used technology in modern molecular biology research, gene sequencing technology has made considerable progress. Conventional gene sequencing technologies include first-generation sequencing technologies represented by Sanger sequencing, next-generation sequencing technologies of high-throughput sequencing, and third-generation sequencing technologies of single molecule sequencing.

First-generation sequencing technologies represented by Sanger sequencing, still considered as the gold standard of gene sequencing, are however extremely costly. Next-generation sequencing technologies of high-throughput sequencing mainly include pyrosequencing, sequencing by synthesis, ion semiconductor sequencing, and sequencing by ligation. Massively parallel sequencing technologies centered on sequencing by synthesis have shown high improvements in both throughput and speed and are currently the mainstream technologies for commercial applications. However, sequencing by synthesis requires not only fluorescent labeling of bases, but also complex laser sources and optical systems, making a sequencing system complex and post-processing of data difficult. In addition, labeling reagents are particularly expensive, limiting the potential for reducing sequencing costs. Further, human-introduced errors are inevitable.

Third-generation sequencing technologies of single molecule sequencing, which are characterized by fast speed and long-read sequencing, have become a new direction pursued by academia and industry. Principles for third-generation sequencing technologies are mainly divided into two categories: optical single-molecule sequencing and electrical single-molecule sequencing.

The representative technology of optical single-molecule sequencing is the zero-mode waveguide technology developed by Pacific Bioscience. The representative technology of electrical single-molecule sequencing is nanopore sequencing, an innovative method developed by Oxford Nanopore Technologies in the UK. Specifically, on a basis of differences in electrical properties of individual bases A, T, C, and G, nanopore sequencing realizes sequence analysis based on differences in electrical signals. However, the zero-mode waveguide technology still requires an excitation light source and an optical system. Inevitably, excitation light becomes background noise in the optical signal. Further, the costs and an error rate are still high. Nanopore sequencing relies on electronic signals excited by an electric field to realize result analysis, but the electronic signals are sensitive to electrical noise. Such noise increases the difficulty in analyzing the electronic signals and improving the signal-to-noise ratio, which in turn limits an improvement of the detection accuracy.

In view of the above problems, the present disclosure is provided.

### SUMMARY

The present disclosure provides a detection structure for solving the technical problem as described in the prior art as follows. In the related art, whether a detection system having an excitation light source and an optical system is used, or a method of generating electronic signals through electrical excitation in technologies such as nanopore sequencing is used, an excitation signal and a detection signal used therein are of the same type, which inevitably leads to background noise, resulting in a technical problem of unsatisfactory detection accuracy.

On the other hand, the present disclosure further provides a detection method based on the detection structure. With a combination of the structure and the method, a better detection result can be achieved. On the other hand, the present disclosure further provides a detection chip and a sensing device. As core elements, the detection chip and the sensing device can facilitate physical isolation of the excitation signal and the detection signal and can be applied in molecular detection and analysis, substance recognition, molecular diagnosis, disease detection, and gene detection and sequencing.

A detection structure includes: a sensing device at least including a detection chip, a fluid tank, and a carrier plate; and a detector configured to capture and analyze a signal generated in the sensing device. The fluid tank is disposed at the carrier plate and forms a cavity with the carrier plate. The detection chip is located in the cavity and at least includes a substrate, and a first electrode and a first circuit that are disposed at the substrate. The first electrode is connected to a second electrode through the first circuit to form an electrical circuit.

In the present disclosure, the sensing device is provided with the detection chip and the fluid tank for enclosing the detection chip. The carrier plate actually serves as a carrier for the detection chip and the fluid tank. The cavity (filled with a sample during a detection) that encloses the detection chip is formed by the carrier plate and the fluid tank. The detection chip includes the substrate, and the first electrode and the second electrode (which is not necessarily disposed at the detection chip) that form the electrical circuit through the first circuit. Therefore, when a voltage is applied to each of the two electrodes for excitation, some components of a to-be-tested sample can be excited to generate a signal of a different type than a form of excitation. The generated signal can be captured by a detector for further analysis and ultimately for analysis of a detection result.

Compared with the related art, the present disclosure differs from a conventional way of directly irradiating a substance with excitation light and generating an optical signal, the conventional way of which inevitably suffers from a defect of optical noise. Further, the present disclosure also differs from a way of generating and analyzing electrical signals through an electrochemical method in technologies such as nanopore sequencing, the way of which results in electrical noise. In the above two conventional ways, the excitation signal and the detection signal used therein are of the same type, which inevitably leads to background noise, increasing the difficulty in signal analysis and affecting the detection accuracy. However, in the present disclosure, the excitation signal and the detection signal are of different types, and thus physical isolation of the excitation signal and the detection signal is fundamentally realized. Therefore, the background noise in the related art can be effectively avoided to improve the detection accuracy.

Optionally, the detector is capable of being integrally disposed in the sensing device or independently disposed outside the sensing device; and/or the second electrode is capable of being integrally disposed at the detection chip or independently disposed outside the detection chip.

Each of the detector and the second electrode may be disposed at a variety of positions to increase applicability of the entire detection structure. When the detector is integrated with the sensing device, it is generally preferable to integrate the detector at the substrate of the detection chip. In addition, the detector may preferably be a light detector.

More specifically, the second electrode may be located in the fluid tank or at the detection chip. The second electrode may be in a circular shape, an elliptic shape, a quadrilateral shape, or a polygonal shape when located at the detection chip, or may be in a cylindrical shape, a polygonal shape, or a sheet-like shape when located in the fluid tank. One or more second electrodes may be provided, such as 10 second electrodes, 50 second electrodes, and 100 second electrodes.

Optionally, the detection structure further includes: a temperature control device configured to control a temperature of a fluid in the sensing device; and a main control device connected to the temperature control device, the sensing device, and the detector, and configured to perform data collection, data storage, and data analysis.

The temperature control device and the main control device are important auxiliary devices of the detection structure for realizing the detection effect. The temperature control device can be configured to control the temperature of the fluid in the sensing device. Preferably, the temperature of the fluid ranges from 0 degree Celsius to 60 degrees Celsius. The detector is configured to detect a signal generated by the sensing device. The main control device is connected to the temperature control device, the sensing device, and the detector. Therefore, the data collection, the data storage, and the data analysis can be realized.

Optionally, the detector is a light detector.

The detector is preferably set as the light detector. For example, the light detector includes, but is not limited to, a charge-coupled device (CCD) camera, a complementary metal oxide semiconductor (CMOS) camera, a scientific complementary metal-oxide-semiconductor (S-CMOS) camera, a photodiode (PD) array, and an avalanche photodiode (APD) array or a photomultiplier tube (PMT) or a silicon photomultiplier (SiPM). The light detector realizes a detection of the light signal using electrochemical excitation during a detection application.

Specifically, through controlling voltages at the electrodes, the light signal is excited using the electrochemical method to realize different types of the excitation signal and the detection signal, which effectively avoids the background noise and reduces the difficulty in analyzing the detection signal, and thus facilitates an improvement in the detection accuracy.

Moreover, it provides better controllability, selectivity, and sensitivity by exciting the light signal using the electrochemical method. In addition, controlling an electrochemical cycle and an electrochemical reaction using the electrodes realizes controllable multiplication and amplification of the light signal, which is further conducive to improving a detection rate and a signal-to-noise ratio of the signal.

Optionally, the first electrodes are arranged in an array at the substrate; and/or an isolation well is provided between every two adjacent first electrodes; and/or the first circuit is disposed in the substrate.

Arranging the first electrodes in an array can allow a large number of first electrodes to be arranged at one and the same substrate to improve the detection efficiency. In addition, the isolation well is provided between the first electrodes and can be configured to eliminate or avoid mutual interference between light signals emitted from adjacent first electrodes simultaneously. A main function of the first circuit is to connect the first electrodes arranged in an array and the second electrode to form the electrical circuit, and to control a potential of the first electrodes in real time based on a detection need, in such a manner that the first electrodes arranged in an array can be controlled uniformly, or the first electrodes in different regions can be time-divisionally controlled. A constant potential or a series of periodic potentials may be applied to the first electrodes through the first circuit based on the detection need.

Optionally, the isolation well has a thickness greater than a thickness of the first electrode, and a gap is provided between an end of the isolation well away from the substrate and a bottom of the fluid tank.

The isolation well is mainly used to eliminate or avoid the mutual interference between the light signals emitted by adjacent first electrodes simultaneously. For the first electrode, the isolation well at two sides of the first electrode actually forms a reaction space of the first electrode. If the thickness of the isolation well is smaller than the thickness of the first electrode, an effect of eliminating or avoiding the interference is limited. In addition, as a reaction cavity for a reaction system, the cavity around the fluid tank and the detection chip should be through and uninterrupted. Therefore, the gap is provided between the end of the isolation well away from the substrate and the bottom of the fluid tank. It should be understood that since the fluid tank is actually disposed at the carrier plate upside down, the bottom of the fluid tank is actually at a top of the structure.

Optionally, the substrate is disposed at the carrier plate, and the fluid tank is connected to the carrier plate; and a second circuit connected to the first circuit is embedded in the carrier plate.

The fluid tank is fixed to the carrier plate to construct the fluid-accommodation cavity for the detection chip. On the other hand, an effect similar to sealing the detection chip is realized by the fluid tank (specifically, a connection of the fluid tank with the carrier plate is a sealed connection, but the reaction cavity formed by the fluid tank and the carrier plate cannot be construed as being isolated from an ambient environment). The second circuit has a function of controlling the first circuit. The first circuit is configured to interconnect the first electrode and the second electrode to form the electrical circuit.

Optionally, the fluid tank has a sample hole for a sample supply to or a sample withdrawal from the cavity.

With the sample hole, the sample supply to or the sample withdrawal from the cavity can be realized. In addition, a quantity, a position, a shape, or the like of the sample hole can be set in a variety of manners. Preferably, the sample hole is in a circular shape. Preferably, more than one sample hole is formed. Preferably, the sample hole is formed at a bottom wall of the fluid tank (i.e., a wall opposite to a tank opening, in which the tank opening is positioned at the carrier plate; and in practice, the tank opening is sealed against and connected to the carrier plate).

A detection method based on the detection structure, includes: attaching a characteristic enzyme to the first electrode, and supplying a to-be-tested sample and a raw material molecule at least modified by a labeled molecule and/or a co-reactive molecule to the cavity available for reaction; and setting a voltage to enable a signal to be generated in a reaction system and captured by the detector, and analyzing the captured signal to obtain a detection result.

The entire detection method includes attaching the characteristic enzyme to the first electrode, constructing the reaction system, and setting and controlling the voltage. Based on specificity of the detection structure and the provided reaction system, an excitation signal and a captured detection signal can be of different types, and thus the physical isolation of the excitation signal and the detection signal can be realized. Therefore, the background noise can be effectively avoided to improve the detection accuracy.

Optionally, the method includes: attaching a nucleic acid polymerase to the first electrode, and supplying a solution containing a to-be-tested nucleic acid sample to the cavity available for reaction; supplying the raw material molecule at least modified by the labeled molecule and/or the co-reactive molecule to the cavity available for reaction; setting a voltage for each of the first electrode and the second electrode, in such a manner that a light signal is emitted from an extension product, complementary to the to-be-tested nucleic acid sample, formed through an action of the raw material molecule and the nucleic acid polymerase; and capturing, by a light detector, the light signal, and obtaining a sequencing result through analyzing the light signal.

It should be understood that in most cases, a solution of the to-be-tested nucleic acid sample and the raw material molecule are supplied to the reaction cavity together as the reaction system. However, in some cases, the solution of the to-be-tested nucleic acid sample and the raw material molecule may also be supplied to the reaction cavity separately. It should also be noted that when the solution containing the to-be-tested nucleic acid sample and the raw material molecule are supplied to the cavity separately, no restriction is exerted on a supply sequence of the solution containing the to-be-tested nucleic acid sample and the raw material molecule.

During the reaction, the raw material molecule has a same basic function as a nucleotide molecule and can be polymerized into a nucleic acid molecular chain, while the labeled molecule carried on the raw material molecule together with the co-reactive molecules on the raw material molecule and/or in the solution can be excited under a predetermined characteristic potential applied by the first electrode, to undergo electrochemical reaction and eventually emit the light signal.

The reaction occurs at a surface of the first electrode. Therefore, the raw material molecule that is not bound to the nucleic acid molecular chain undergoes no luminescence reaction. Typically, four nucleotides are required, i.e., nucleotides with A, T, C, and G bases. The four nucleotides are modified with four different labeled molecules, respectively. The four nucleotides are modified with four different labeled molecules. The different labeled molecules may have different characteristic potentials, or emit light signals having different wavelengths, or be provided with a combination of the above-mentioned features.

Optionally, the method further includes: cleaving a labeled molecule and/or a co-reactive molecule in the extension product by the nucleic acid polymerase to form a free molecule.

Optionally, the labeled molecule includes a metal-organic complex and a derivative thereof, a polycyclic aromatic hydrocarbon compound and a derivative thereof, or a hydrazide compound and a derivative thereof; and/or the co-reactive molecule includes oxalate, peroxysulfate, tripropylamine, or hydrogen peroxide; and/or the raw material molecule includes a nucleotide.

Preferably, the raw material molecule may be a nucleotide modified by one or more labeled molecules, or a nucleotide modified by both one or more labeled molecules and one or more co-reactive molecules.

A detection chip at least includes: a substrate; a first electrode disposed at the substrate; and a first circuit, the first electrode being connected to a second electrode through the first circuit to form an electrical circuit.

The detection chip includes the substrate, the first electrode, and the first circuit. The first electrode is disposed at the substrate. Therefore, a site for electrochemical reaction can be formed at the first electrode. Since the first circuit is capable of connecting the first electrode to the second electrode, in a case where the reaction system is available, the electrochemical reaction can be realized through applying the voltage to each of the two electrodes, and the reaction system is induced to generate a non-electrical signal (e.g., the light signal) that can be collected and further analyzed. In this way, the physical isolation of the excitation signal and the detection signal can be realized.

Optionally, the first electrode is connected, through the first circuit, to the second electrode, which is capable of being integrally disposed at the detection chip or independently disposed outside the detection chip, to form the electrical circuit.

Optionally, the detection chip further includes the second electrode disposed at the substrate.

The second electrode serves to form the electrical circuit with the first electrode through the first circuit and may be set at any position as desired. For example, the second electrode may be disposed at the substrate as a part of the detection chip, or disposed at another position (e.g., the carrier plate) independent of the detection chip.

Optionally, the substrate includes a semiconductor substrate, an insulator substrate, a semiconductor-on-insulator substrate, or a printed circuit board; and/or the first electrode or the second electrode includes a metallic electrode, a multilayer metallic composite electrode, a silver chloride electrode, an indium tin oxide electrode, a carbon-based material electrode, or a composite electrode of a carbon-based material and a metal.

The substrate has a function of providing a carrier for the first electrode and the first circuit and may be of various types that can be determined based on application scenarios and demands. Similarly, the first electrode or the second electrode may also be of various types.

A sensing device includes: the above-mentioned detection chip; a carrier plate; and a fluid tank disposed at the carrier plate and forming a cavity with the carrier plate, the detection chip being located in the cavity.

As an important device of the entire detection structure, the sensing device is a site where the electrochemical reaction takes place and is composed of the detection chip, the carrier plate, and the fluid tank. The detection chip is disposed in the cavity formed by the carrier plate and the fluid tank. In this way, the detection chip and the reaction cavity in which the detection chip is located constitute the reaction site of the reaction system. The signal captured and analyzed by the detector is generated through applying the voltage to each of the two electrodes.

Optionally, the sensing device further includes a detector disposed at the substrate of the detection chip and configured to capture a signal generated in the sensing device.

When the detector is integrated with the sensing device, it is generally preferable to integrate the detector and the sensing device at the substrate of the detection chip, which will make the entire sensing device simple and integrated. Preferably, the detector is the light detector, which facilitates capturing of the light signal in the sensing device.

In summary, in the present disclosure, the detection chip is the core component, and provides a basic guarantee for the electrochemical reaction to generate the non-electrical signal. The sensing device further provides the reaction site such as the reaction cavity on a basis of the detection chip. The detection structure, which integrates the detection chip and the sensing device, excites the light signal using the electrochemical method, and obtains a sequencing light signal through applying electrical excitation, in such a manner that the physical isolation of the excitation signal and the detection signal is realized. Therefore, the background noise in the related art can be effectively avoided to improve the detection accuracy.

In the present disclosure, the light signal is excited using the electrochemical method. Two conventional methods include one method exciting the light signal using the excitation light source and the optical system, and another method of nanopore sequencing relying on electronic signals excited by an electric field to realize result analysis. Compared with the above two conventional methods, the present disclosure realizes the physical isolation of the excitation signal and the detection signal, and thus has an advantage of noise reduction.

In the present disclosure, exciting the light signal using the electrochemical method provides better controllability, selectivity, and sensitivity. Controlling the electrochemical cycle and the electrochemical reaction using the electrodes can realize controllable multiplication and amplification of the light signal, which is conducive to improving the detection rate and the signal-to-noise ratio of the signal.

In the present disclosure, the use of the detector (photodetector) integrated in the sensing device assists in improving an integration level of the detection structure and reducing a volume of the detection system.

The sensing device, the detection structure, and the detection method of the present disclosure can be widely applied in molecular detection and analysis, substance recognition, molecular diagnosis, disease detection, gene detection and sequencing, and the like, and therefore have promising application prospects.

### BRIEF DESCRIPTION OF THE DRAWINGS

In order to clearly explain embodiments of the present disclosure or technical solutions in the related art, drawings used in the description of the embodiments or the related art are briefly described below. As can be easily understood, the drawings as described below are some embodiments of the present disclosure. Based on these drawings, other drawings can be obtained by those skilled in the art without creative effort.

FIG. 1a is a schematic planar structural view of a detection chip according to an embodiment of the present disclosure.

FIG. 1b is a schematic sectional structural view of a detection chip according to an embodiment of the present disclosure.

FIG. 1c is another schematic sectional structural view of a detection chip according to an embodiment of the present disclosure.

FIG. 1d is yet another schematic sectional structural view of a detection chip according to an embodiment of the present disclosure.

FIG. 2a is a schematic sectional structural view of a sensing device according to an embodiment of the present disclosure.

FIG. 2b is another schematic sectional structural view of a sensing device according to an embodiment of the present disclosure.

FIG. 3 is a schematic view of a system architecture of a detection structure according to an embodiment of the present disclosure.

FIG. 4 is a schematic view of a raw material molecule according to an embodiment of the present disclosure.

FIG. 5 is a schematic view of a signal detection according to an embodiment of the present disclosure.

FIG. 6 is a schematic view of a detection chip provided with first electrodes according to an embodiment of the present disclosure.

FIG. 7 and FIG. 8 are a schematic diagram of simultaneously applying a periodic potential to all first electrodes under a condition of an arrangement scheme of the first electrodes in FIG. 6 and a schematic diagram of time-divisionally applying a potential to different first electrodes under the condition of the arrangement scheme of the first electrodes in FIG. 6, respectively.

### REFERENCE NUMERALS OF THE ACCOMPANYING DRAWINGS

detection chip 000;
substrate 001: first electrode 002, first circuit 003, second electrode 004, isolation well 005;
sensing device 100;
fluid tank 101: carrier plate 102; cavity 103; second circuit 104; sample hole 105;
detection structure 200;
detector 201: temperature control device 203; main control device 204;
raw material molecule 300;
nucleotide 301: labeled molecule 302; co-reactive molecule 303;
nucleic acid polymerase 400; to-be-tested nucleic acid molecule 500.

### DETAILED DESCRIPTION

In order to make the above and other features and advantages of the present disclosure more apparent, the present disclosure is described in detail below with reference to the accompanying drawings. It should be understood that the embodiments described herein are only used to explain to those skilled in the art, and are merely illustrative rather than restrictive.

In the description of the present disclosure, it should be understood that, the orientation or the position indicated by terms such as “center”, “longitudinal”, “lateral”, “length”, “width”, “thickness”, “over”, “below”, “front”, “rear”, “left”, “right”, “vertical”, “horizontal”, “top”, “bottom”, “inner”, “outer”, “clockwise”, “anti-clockwise”, “axial”, “radial”, and “circumferential” should be construed to refer to the orientation and the position as shown in the drawings, and is only for the convenience of describing the present disclosure and simplifying the description, rather than indicating or implying that the pointed device or element must have a specific orientation, or be constructed and operated in a specific orientation, and therefore cannot be understood as a limitation of the present disclosure.

In addition, terms “first” and “second” are only used for descriptive purposes, and cannot be understood as indicating or implying relative importance or implicitly indicating the number of indicated technical features. Therefore, the features associated with “first” and “second” may explicitly or implicitly include at least one of the features. In the description of the present disclosure, “plurality” means at least two, such as two, three etc., unless otherwise specifically defined.

In the present disclosure, unless otherwise clearly specified and limited, terms such as “install”, “connect”, “connect to”, “fix” and the like should be understood in a broad sense. For example, it may be a fixed connection or a detachable connection or connection as one piece: mechanical connection or electrical connection: direct connection or indirect connection through an intermediate: internal communication of two components or the interaction relationship between two components, unless otherwise clearly limited. For those skilled in the art, the specific meaning of the above-mentioned terms in the present disclosure can be understood according to specific circumstances.

In the present disclosure, unless expressly stipulated and defined otherwise, the first feature “on” or “under” the second feature may include that the first feature is in direct contact with the second feature, or further include that the first and second features are in indirect contact through an intermediate. Moreover, the first feature “above” the second feature may mean that the first feature is directly above or obliquely above the second feature, or simply mean that the level of the first feature is higher than that of the second feature. The first feature “below” the second feature may mean that the first feature is directly below or obliquely below the second feature, or simply mean that the level of the first feature is smaller than that of the second feature.

Reference throughout this specification to “an embodiment”, “some embodiments”, “an example”, “a specific example”, or “some examples” means that a particular feature, structure, material, or characteristic described in connection with the embodiment or example is included in at least one embodiment or example of the present disclosure. The appearances of the above phrases in various places throughout this specification are not necessarily referring to the same embodiment or example. Further, the particular features, structures, materials, or characteristics may be combined in any suitable manner in one or more embodiments or examples. In addition, different embodiments or examples and features of different embodiments or examples described in the specification may be combined by those skilled in the art without mutual contradiction.

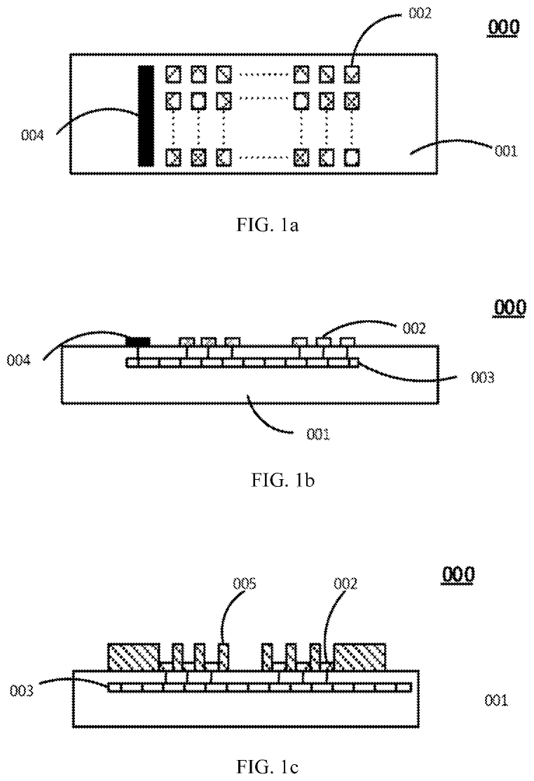

As illustrated in FIG. 1a to FIG. 1d, in a specific embodiment of the present disclosure, a detection chip 000 is provided. The detection chip 000 includes a substrate 001, a first electrode 002, and a first circuit 003. The first electrode 002 is disposed at the substrate 001, and is connected to a second electrode 004 through the first circuit 003 to form an electrical circuit.

Other embodiments of the detection chip 000 may also be a further limitation or addition performed on one or a combination of the following embodiments based on the above-mentioned embodiments.

For example, as illustrated in FIG. 1a, FIG. 1b, and FIG. 1d, the detection chip 000 includes the second electrode 004. The second electrode 004 is disposed at the substrate 001. The substrate 001 includes a semiconductor substrate, an insulator substrate, a semiconductor-on-insulator substrate, or a printed circuit board. The first electrode 002 or the second electrode 004 includes a metallic electrode, a multilayer metallic composite electrode, a silver chloride electrode, an indium tin oxide electrode, a carbon-based material electrode, or a composite electrode of a carbon-based material and a metal. The first electrodes 002 are arranged in an array at the substrate 001. An isolation well 005 is provided between every two adjacent first electrodes 002; and/or the first circuit 003 is disposed in the substrate 001.

It should be understood that FIG. 1c differs from FIG. 1a, FIG. 1b, and FIG. 1d in that in the embodiment of the detection chip 000 illustrated in FIG. 1c, second electrode 004 is not disposed at the substrate 001. Also, the detection chip 000 illustrated in each of FIG. 1c and FIG. 1d mainly differs from that of FIG. 1a and FIG. 1b in that the isolation well 005 is provided.

In some embodiments, the substrate 001 is a semiconductor, e.g., silicon: or an insulator, e.g., quartz glass: or a semiconductor-on-insulator, e.g., silicon-on-insulator: or a printed circuit board, i.e., so-called PCB board.

The substrate 001 may be in various shapes. In a preferred embodiment, generally, the substrate 001 is in a rectangular shape and has a thickness ranging from 100 micrometers to 10 millimeters and a length or a width ranging from 0.5 millimeters to 500 millimeters.

In some embodiments, each of the first electrode 002 and the second electrode 004 may be made of silver and silver chloride: or an inert metal, such as platinum, gold, and palladium: or a multilayer composite metal, such as titanium-platinum, nickel-platinum, titanium-gold, nickel-gold, titanium-palladium, and nickel-palladium: or a carbon-based material, such as graphene or a carbon nanotube: or a composite of a carbon-based material and a metal, such as graphene and platinum, graphene and gold, and the like: or indium tin oxide (ITO).

The first electrode 002 may have a topology structure in a circular shape, an elliptic shape, a quadrilateral shape, or a polygonal shape. The first electrode 002 has a thickness generally ranging from 1 nanometer to 100 micrometers, which is determined by taking into account performance and manufacturing costs in actual design and manufacturing. Preferably, the first electrode 002 has the thickness of 200 nanometers. The first electrode 002 has a diameter or a long axis or a short axis ranging from 1 nanometer to 1 micrometer.

In one and the same detection chip 000, one first electrode 002 or a plurality of first electrodes 002 arranged in an array may be provided. The quantity of arrays may be determined based on a design demand, and may be 10³, 10⁹, 10¹², or the like. A spacing between two adjacent first electrodes 002 preferably ranges from 1 nanometer to 10 micrometers.

In some embodiments, the isolation well 005 may be made of a metallic material, an organic material, or a semiconductor material such as silicon oxide. The isolation well 005 may be in a circular shape, an elliptic shape, a quadrilateral shape, or a polygonal shape, and have a structural linewidth ranging from 1 nanometer to 10 micrometers and a height (thickness) ranging from 10 nanometers to 100 micrometers.

In some preferred embodiments, the second electrode 004 is located at the detection chip 000 and may be in a circular shape, an elliptic shape, a quadrilateral shape, or a polygonal shape.

In a specific embodiment of the present disclosure, a sensing device 100 is provided. The sensing device 100 includes the detection chip 000 according to any of the above-mentioned embodiments, a fluid tank 101, and a carrier plate 102. The fluid tank 101 is disposed at the carrier plate 102 and forms a cavity 103 with the carrier plate 102. The detection chip 000 is located in the cavity 103.

Other embodiments of the sensing device 100 of the present disclosure may also be a further limitation or addition performed on one or a combination of the following embodiments based on the above-mentioned embodiments.

For example, the isolation well 005 has the thickness greater than the thickness of the first electrode 002. A gap is provided between an end of the isolation well 005 away from the substrate 001 and a bottom of the fluid tank 101. The substrate 001 is disposed at the carrier plate 102. The fluid tank 101 is connected to the carrier plate 102. A second circuit 104 connected to the first circuit 003 is embedded in the carrier plate 102. The fluid tank 101 has a sample hole 105 for a sample supply to or a sample withdrawal from the cavity 103. Alternatively, the sensing device 100 further includes a detector 201, which is disposed at the substrate 001 of the detection chip 000 and configured to capture a signal generated in the sensing device 100.

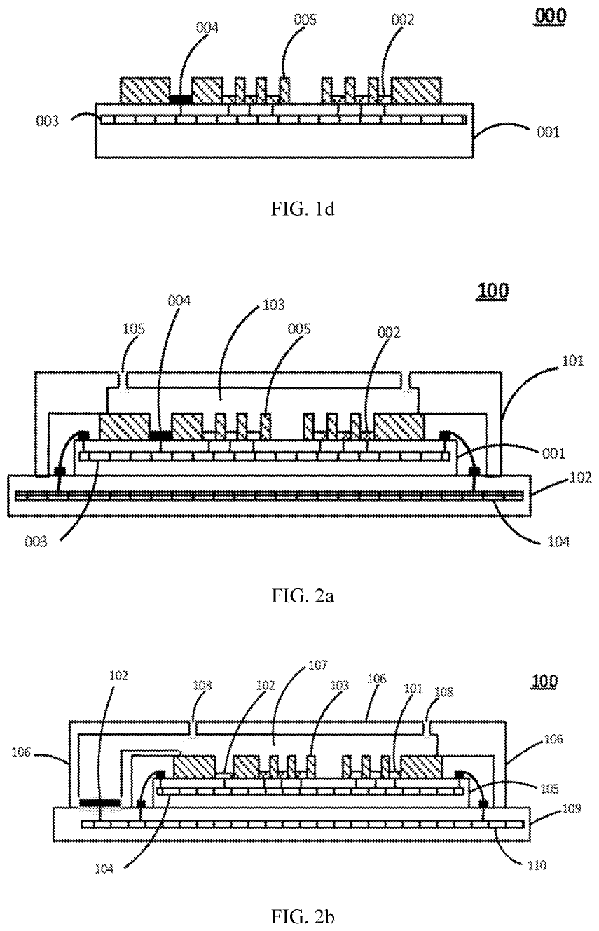

In some other embodiments, as illustrated in FIG. 2a, the second electrode 004 in the sensing device 100 is located at the detection chip 000. In some other embodiments, as illustrated in FIG. 2b, the second electrode 004 in the sensing device 100 is located in the fluid tank 101. In particular, the second electrode 004 in the sensing device 100 may be disposed at the carrier plate 102. A main difference between the sensing device 100 illustrated in FIG. 2a and the sensing device 100 illustrated in FIG. 2b lies in different positions of the second electrodes 004. In both structures, the second electrode 004 can realize an electrical connection through a reaction system supplied to the cavity 103, and is ultimately connected to the first circuit 003 or the second circuit 104.

In some embodiments, the carrier plate 102 may be a printed circuit board, or be made of a plastic material, a ceramic material, or the like. As illustrated in FIG. 2a and FIG. 2b, the second circuit 104 is disposed in the carrier plate 102 and configured to control the first circuit 003. The detection chip 000 is fixed to the carrier plate 102. The first circuit 003 at the detection chip 000 is connected to the second circuit 104 at the carrier plate 102. The fluid tank 101 may be made of a non-conductive material such as plastic, ceramic, or the like. The first electrode 002 is connected to the second electrode 004 through the cavity 103. The electrical circuit is formed between the first electrode 002 and the second electrode 004 when the cavity is filled with a fluid (reaction system).

In some specific embodiments, the first electrode 002 has a length or the long axis ranging from 1 nanometer to 1 micrometer. The first electrode 002 has a width or the short axis ranging from 1 nanometer to 1 micrometer. The first electrode 002 has the thickness (height) ranging from 1 nanometer to 100 micrometers. The spacing between two adjacent first electrodes in an array of first electrodes 002 ranges from 1 nanometer to 10 micrometers.

When located at the detection chip 000, the second electrode 004 has a length or a long axis ranging from 1 nanometer to 100 millimeters, a width or a short axis ranging from 1 nanometer to 100 millimeters, and a thickness ranging from 1 nanometer to 100 micrometers. When not located at the detection chip 000, the second electrode 004 has the length or a diameter ranging from 1 nanometer to 100 millimeters, the width or the diameter ranging from 1 nanometer to 100 millimeters, and a height or the thickness ranging from 1 nanometer to 10 millimeters.

The substrate 001 has the thickness ranging from 100 micrometers to 10 millimeters, the length ranging from 0.5 millimeters to 500 millimeters, and the width ranging from 0.5 millimeters to 500 millimeters.

The carrier plate 102 has a thickness ranging from 100 micrometers to 10 millimeters, a length ranging from 0.5 millimeters to 500 millimeters, and a width ranging from 0.5 millimeters to 500 millimeters.

The fluid tank 101 has a length ranging from 0.5 millimeters to 500 millimeters and a width ranging from 0.5 millimeters to 500 millimeters. A cavity formed by the fluid tank 101 and the chip has a height ranging from 1 micrometer to 10 millimeters.

In some embodiments, the fluid tank 101 is fixed to the detection chip 000 and the carrier plate 102, and consequently, the cavity 103 for accommodating a fluid solution is formed between the detection chip 000 and the fluid tank 101. One or more sample holes 105 for a solution supply or a solution withdrawal is formed at the fluid tank 101.

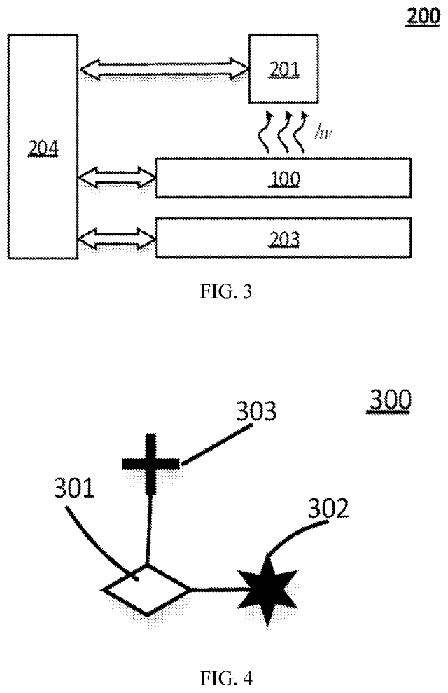

As illustrated in FIG. 2a, FIG. 2b, and FIG. 3, in a specific embodiment of the present disclosure, a detection structure 200 is provided. The detection structure 200 includes: the sensing device 100 including the detection chip 000, the fluid tank 101, and the carrier plate 102; and the detector 201 configured to capture and analyze a signal generated in the sensing device 100. The fluid tank 101 is disposed at the carrier plate 102 and forms the cavity 103 with the carrier plate 102. The detection chip 000 is located in the cavity 103 and at least includes the substrate 001 and the first electrode 002 and the first circuit 003 that are disposed at the substrate 001. The first electrode 002 is connected to the second electrode 004 through the first circuit 003 to form the electrical circuit.

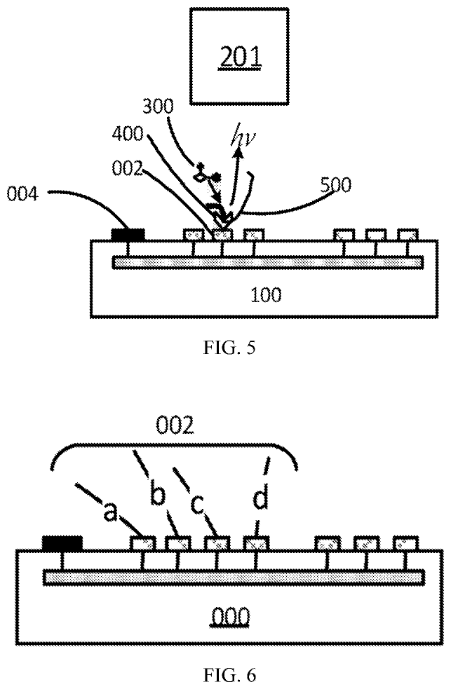

Each of the detection chip 000 and the sensing device 100 may be implemented as the solution described in any of the above-mentioned embodiments. In addition, in some embodiments, the detector 201 is integrally disposed in the sensing device 100 (not illustrated in the figures); or independently disposed outside the sensing device 100 (for example, in FIG. 5, the sensing device 100 and the detector 201 are arranged opposite to each other). Further, the second electrode 004 may be integrally disposed at the detection chip 000 or independently disposed outside the detection chip 000.

In other preferred embodiments, the detection structure 200 further includes a temperature control device 203 and a main control device 204.

The temperature control device 203 is configured to control a temperature of a fluid in the sensing device 100. The main control device 204 is connected to the temperature control device 203, the sensing device 100, and the detector 201, and configured to perform data collection, data storage, and data analysis.

The temperature of the fluid is controlled to range from 0 degree Celsius to 60 degrees Celsius. The detector 201 is preferably a light signal detector configured to detect the light signal generated by the sensing device 100.

The temperature control device 203 generally adopts a semiconductor temperature control module based on Proportional Integral Derivative (PID) logic control, which is a mature temperature control technology, and thus details thereof will be omitted here.

The detector 201 may be a light detector such as a CCD camera, a CMOS camera, an S-CMOS camera, a PD array, and an APD array or a PMT or an SiPM. The detector 201 can be configured to detect the light signal emitted from the sensing device 100 during a detection and transmit the light signal to the main control device 204.

In a specific embodiment of the present disclosure, a detection method is provided. The method includes: attaching a characteristic enzyme to the first electrode 002, and supplying a to-be-tested sample and a raw material molecule 300 at least modified by a labeled molecule 302 and/or a co-reactive molecule 303 to the cavity 103 available for reaction; and setting a voltage to enable a signal to be generated in a reaction system and captured by the detector 201, and analyzing the captured signal to obtain a detection result.

Reference can be made to FIG. 5, which illustrates a schematic view of a signal detection implemented by the detection structure 200 according to an embodiment of the present disclosure. In some more specific embodiments of the detection method, e.g., in embodiments where the detection method is specifically applied in nucleic acid sequencing, the method specifically includes the following operations.

A nucleic acid polymerase 400 is attached to the first electrode 002, and a solution containing a to-be-tested nucleic acid sample is supplied to the cavity 103 available for reaction. The raw material molecule 300 at least modified by the labeled molecule 302 and/or the co-reactive molecule 303 is supplied to the cavity 103 available for reaction.

It should be understood that the solutions here can be supplied in various ways. For example, the solution of the to-be-tested nucleic acid sample and a solution containing the raw material molecule 300 modified by the labeled molecule 302 and/or the co-reactive molecule 303 may be supplied as a whole or may be supplied separately.

The way in which the solution of the to-be-tested nucleic acid sample and the solution containing the raw material molecule 300 modified by the labeled molecule 302 and/or the co-reactive molecule 303 are supplied separately can be interpreted as that each of the solution of the to-be-tested nucleic acid sample in a solution form and the solution of the raw material molecule 300 is supplied to the reaction cavity alone. No restriction is exerted on a supply sequence of the solution of the to-be-tested nucleic acid sample and the solution of the raw material molecule 300.

A voltage is set for each of the first electrode 002 and the second electrode 004, in such a manner that a light signal is emitted from an extension product, complementary to the to-be-tested nucleic acid sample, formed through an action of the raw material molecule 300 and the nucleic acid polymerase 400.

The light signal is captured by the detector 201 (specifically, the light detector). A sequencing result is obtained through analyzing the light signal.

The labeled molecule 302 has electrochemiluminescent activity, and can release the light signal due to electrochemical reaction under an action of the first electrode 002 and the co-reactive molecule 303. Different labeled molecules 302 may have different characteristic potentials, or emit light signals having different wavelengths, or be provided with both of the above-mentioned two features. For ease of understanding, FIG. 4 illustrates a schematic view of a modified raw material molecule 300.

In some embodiments, the labeled molecule 302 may be a metal-organic complex and a derivative thereof, such as bipyridine ruthenium, bipyridine iridium, bipyridine osmium, or the like. In other embodiments, the labeled molecule 302 may be a polycyclic aromatic hydrocarbon compound and a derivative thereof, such as 9,10-diphenylanthracene. Further, the labeled molecule 302 may be a hydrazide compound and a derivative thereof, such as luminol.

The co-reactive molecule 303 may be oxalate, peroxysulfate, tripropylamine, hydrogen peroxide, or the like. The raw material molecule 300 may be a nucleotide 301 modified by one or more labeled molecules 302, or a nucleotide 301 modified by one or more labeled molecules 302 and one or more co-reactive molecules 303 simultaneously. In the raw material molecule 300, a nucleotide 301 molecule carrying different bases is modified by different labeled molecules 302 and/or co-reactive molecules 303. It should be understood that the nucleotide 301 molecule modified by different labeled molecules 302 or co-reactive molecules 303 may be excited with light signals having different wavelengths or have different characteristic potentials.

Further, in a specific embodiment of the present disclosure, a method for analyzing a nucleic acid molecule sequence is provided. The method includes the following operations.
At S-1, the detection structure 200 of the present disclosure is provided.
At S-2, the nucleic acid polymerase 400 is attached to the first electrode 002.
At S3, a solution containing the to-be-tested nucleic acid molecule 500 is supplied to the cavity.
At S4, the raw material molecule 300 at least modified by one labeled molecule 302 and/or co-reactive molecule 303 is supplied to the cavity.

In some reactions, if necessary, one or more additional co-reactive molecules 303 may continue to be supplied to the formed reaction system to fulfill needs of the reactions.
At S5, the raw material molecule 300 is synthesized onto the to-be-tested nucleic acid molecule 500 under an action of the nucleic acid polymerase 400, in such a manner that the raw material molecule 300 becomes the extension product complementary to the to-be-tested nucleic acid molecule 500.
At S6, a specific potential is set for each of the first electrode 002 and the second electrode 004.
At S7, under excitation of the characteristic potential and the co-reactive molecule 303, the labeled molecule 302 on the extension product undergoes an electrochemical reaction and emits a light signal.
At S8, the light signal is captured by the detector 201, converted into an electrical signal, and transmitted to the main control device 204.
At S9, the labeled molecule 302 on the extension product is further cleaved by the nucleic acid polymerase 400 and supplied to the solution as a free molecule.
At S10, a series of light signal information during synthesis of the to-be-tested nucleic acid molecule 500 is obtained through repeating operations at S5 to S8, and sequence information of the nucleic acid molecule is obtained through analysis.

Through analyzing information such as a wavelength of a luminescence signal or the characteristic potential corresponding to the nucleotide 301 when the nucleotide 301 emits the light signal, a category of the labeled molecule 302 or the co-reactive molecule 303 modifying the nucleotide 301 can be obtained, and the sequence information of the to-be-tested nucleic acid molecule 500 could be further obtained.

In a specific application, according to an embodiment of the present disclosure, a constant potential or a periodic potential may be applied to the array of first electrodes 002 in one and the same sensing device 100 simultaneously. In another embodiment of the present disclosure, as illustrated in FIG. 6 to FIG. 8, a potential may be applied to only some of the first electrodes 002 while no potential is applied to the other electrodes of the first electrodes 002.

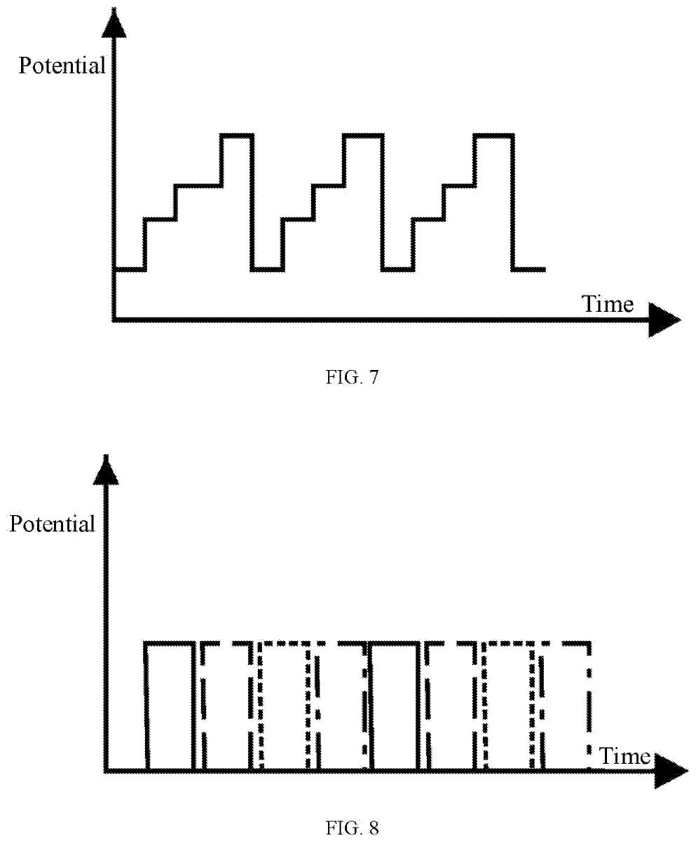

FIG. 6 is a schematic view of the detection chip 000 provided with first electrodes 002 according to an embodiment of the present disclosure. On this basis, FIG. 7 is a schematic diagram of simultaneously applying a periodic potential to all first electrodes 002 under a condition of an arrangement scheme of the first electrodes 002 in FIG. 6, and FIG. 8 is a schematic diagram of time-divisionally applying a potential to different first electrodes 002 under the condition of the arrangement scheme of the first electrodes 002 in FIG. 6.

Although embodiments of the present disclosure have been shown and described above, it should be understood that the above embodiments are merely exemplary, and cannot be construed to limit the present disclosure. For those skilled in the art, changes, alternatives, and modifications can be made to the embodiments without departing from the scope of the present disclosure.

1. A detection structure, comprising a sensing device at least comprising a detection chip, a fluid tank, and a carrier plate; and
a detector configured to capture and analyze a signal generated in the sensing device, wherein:
the fluid tank is disposed at the carrier plate and forms a cavity with the carrier plate, the detection chip is located in the cavity and at least comprises a substrate, and a first electrode and a first circuit that are disposed at the substrate, and
the first electrode being connected to a second electrode through the first circuit to form an electrical circuit.

2. The detection structure according to claim 1, wherein:
the detector is capable of being integrally disposed in the sensing device or independently disposed outside the sensing device; and/or
the second electrode is capable of being integrally disposed at the detection chip or independently disposed outside the detection chip.

3. The detection structure according to claim 1, further comprising:
a temperature control device configured to control a temperature of a fluid in the sensing device; and
a main control device connected to the temperature control device, the sensing device, and the detector and configured to perform data collection, data storage, and data analysis.

4. The detection structure according to claim 1, wherein the detector is a light detector.

5. The detection structure according to claim 1, wherein:
the first electrodes are arranged in an array at the substrate; and/or
an isolation well is provided between every two adjacent first electrodes; and/or
the first circuit is disposed in the substrate.

6. The detection structure according to claim 5, wherein the isolation well has a thickness greater than a thickness of the first electrode, and a gap is provided between an end of the isolation well away from the substrate and a bottom of the fluid tank.

7. The detection structure according to claim 1, wherein:
the substrate is disposed at the carrier plate, and the fluid tank is connected to the carrier plate; and
a second circuit connected to the first circuit is embedded in the carrier plate.

8. The detection structure according to claim 7, wherein the fluid tank has a sample hole for a sample supply to or a sample withdrawal from the cavity.

9. A detection method based on the detection structure according to claim 1, the method comprising:
attaching a characteristic enzyme to the first electrode, and supplying a to-be-tested sample and a raw material molecule at least modified by a labeled molecule and/or a co-reactive molecule to the cavity available for reaction; and
setting a voltage to enable a signal to be generated in a reaction system and captured by the detector, and analyzing the captured signal to obtain a detection result.

10. The method according to claim 9, comprising:
attaching a nucleic acid polymerase to the first electrode, and supplying a solution containing a to-be-tested nucleic acid sample to the cavity available for reaction;
supplying the raw material molecule at least modified by the labeled molecule and/or the co-reactive molecule to the cavity available for reaction;
setting a voltage for each of the first electrode and the second electrode, in such a manner that a light signal is emitted from an extension product, complementary to the to-be-tested nucleic acid sample, formed through an action of the raw material molecule and the nucleic acid polymerase; and
capturing, by a light detector, the light signal, and obtaining a sequencing result through analyzing the light signal.

11. The method according to claim 10, further comprising:
cleaving a labeled molecule and/or a co-reactive molecule in the extension product by the nucleic acid polymerase to form a free molecule.

12. The method according to claim 11, wherein:
the labeled molecule comprises a metal-organic complex and a derivative thereof, a polycyclic aromatic hydrocarbon compound and a derivative thereof, or a hydrazide compound and a derivative thereof; and/or
the co-reactive molecule comprises oxalate, peroxysulfate, tripropylamine, or hydrogen peroxide; and/or
the raw material molecule comprises a nucleotide.

13. A detection chip, at least comprising:
a substrate;
a first electrode disposed at the substrate; and
a first circuit, wherein the first electrode is connected to a second electrode through the first circuit to form an electrical circuit.

14. The detection chip according to claim 13, further comprising:
the second electrode disposed at the substrate.

15. The detection chip according to claim 13, wherein:
the substrate comprises a semiconductor substrate, an insulator substrate, a semiconductor-on-insulator substrate, or a printed circuit board; and/or
the first electrode or the second electrode comprises a metallic electrode, a multilayer metallic composite electrode, a silver chloride electrode, an indium tin oxide electrode, a carbon-based material electrode, or a composite electrode of a carbon-based material and a metal.

16. A sensing device, comprising:
the detection chip according to claim 13;
a carrier plate; and
a fluid tank disposed at the carrier plate and forming a cavity with the carrier plate, wherein the detection chip is located in the cavity.

17. The sensing device according to claim 16, further comprising:
a detector disposed at the substrate of the detection chip and configured to capture a signal generated in the sensing device.
